(19) United States
(12) Patent Application Publication
(10) Pub. No.: US 2021/0353348 A1
(43) Pub. Date: Nov. 18, 2021

(54) Bone Cement Mixing System and Apparatus

(71) Applicant: Fidelis Partners, LLC, Cheyenne, WY (US)

(72) Inventors: Christopher Pedicini, Franklin, TN (US); Joshua Pedicini, Franklin, TN (US)

(73) Assignee: Fidelis Partners, LLC, Cheyenne, WY (US)

(21) Appl. No.: 17/125,247

(22) Filed: Dec. 17, 2020

Related U.S. Application Data
(60) Provisional application No. 63/024,654, filed on May 14, 2020.

(51) Int. Cl. A61B 17/88; B01F 15/00; B01F 13/00; B01F 7/00

(52) U.S. Cl. CPC A61B 17/8833; B01F 15/00376; B01F 15/00175; A61B 2017/00084; B01F 13/0028; B01F 7/00291; A61B 2017/8838; B01F 15/00201

(57) ABSTRACT

An apparatus for mixing bone cement for use in orthopedic surgeries includes a mixing region and an agitator for mix the cement ingredients. A temperature sensing means of apparatus determines when that the cement mix can no longer be used. The apparatus can be operated or acted on by a power tool for the mixing of the bone cement. The power tool and/or mixing apparatus may include a mechanical torque limiter that inhibits the agitator as a result of mixing torque exceeding a threshold value. A timer of the apparatus may be started by a sensor of the apparatus or by the user in response to a mechanical torque value. When the timer is complete, a cue by the apparatus signals to the user that the mixed cement is ready and able to be applied.

### CROSS-REFERENCE TO RELATED APPLICATIONS

The present disclosure claims priority under 35 U.S.C. § 119 on U.S. Provisional Application Ser. No. 63/024,654 filed on May 14, 2020, the disclosure of which is incorporated by reference.

### FIELD OF DISCLOSURE

The present disclosure relates to the preparation of adhesive compounds for use in orthopedic surgeries, and more specifically, to the mixing of an at least two-component bone cement for use in implant fixation in surgeries, such as (but not necessarily limited to) Total Hip Arthroplasty and Total Knee Arthroplasty.

### BACKGROUND OF THE DISCLOSURE

This disclosure relates to an improvement of bone cement mixing techniques and systems. Bone cement is used in the field of surgery to assist in fixing a surgical implant to a patient's bone, for example.

In general, polymethyl methacrylate (PMMA) is the most commonly found bone cement in orthopedic and trauma surgery. The cement product is formed by mixing a liquid methacrylate (MMA) monomer and a powder MMA co-polymer (it being understood that this liquid and powder also contains other factors such as accelerators and initiators that enable adequate polymerization). This mixing is only done in the operating room when, in the example of orthopedic knee or hip replacement surgery, the implant is ready to be fixed in the bone. Thus, proper preparation of the cement occurs under very constrained conditions, creating a need for precision in the mixing process.

Most bone cement mixing systems currently in use rely on a surgeon's assistant to manually mix the bone cement powder and liquid monomer. After this manual mixing is done, the surgeon's assistant is required to perform a qualitative test (commonly called the “fingertip test”) to determine when the bone cement is ready to be applied. This test is imprecise at best and further suffers from subjectivity and variance from the fact that surgeons often have multiple different assistants who prepare the bone cement, each of whom may have a different technique/mixing speed that results in differing “ready” times for the bone cement. Automated mixing systems exist in the prior art but they too are subject to the qualitative and unreliable “fingertip test” that must be performed manually. In addition, different types of gloves have been shown to result in “ready” times varying by as much as 250%. This can be related to surgeon feel, glove thickness, glove material and glove surface, amongst other things.

Depending on the surgeon's particular assistant, the prepared bone cement may be ready for use from anywhere between two to eight minutes after mixing is completed. The bone cement only stays in its “ready” state for about three minutes (which ready state is also known as “working time” or “application time”), and if the surgeon does not have the bone and implant prepared in that amount of time then the bone cement hardens and can no longer be used. Alternatively, a situation can arise where the bone and implant are ready, and the cement is still in its “waiting” phase. The waiting phase is similarly undesirable for at least the reason that surgery time is expensive, and efficiency is of utmost important in the operating room.

All of the currently available systems and methods for bone cement mixing suffer from one or more of the following disadvantages: ambiguous and imprecise indicator of when the mix is ready (from the fingertip test), cement nonuniformity, mixing techniques differing and dependent on the particular different surgeon's assistant that is performing the mixing at any one time (which can result in differing and unpredictable ready/preparation times), and the fact that the process is time-consuming in and of itself.

### SUMMARY OF THE DISCLOSURE

In view of the foregoing disadvantages inherent in the prior art, the purpose of the present disclosure is to provide a bone cement mixing system and apparatus that provide a uniform mix quickly and with clear indicators of when the mix is ready for use and when the mix is no longer useable. It is understood that PMMA and bone cement refer to the same compound, however, it should be apparent that the apparatus described herein may be used with other multicomponent adhesive materials and compounds used in orthopedic surgery.

In an embodiment, a mixing apparatus uses an asymmetrical mixing pattern to achieve a more uniform mix. In an embodiment the apparatus comprises a mixing region (such as a reservoir, container or bowl, for example) in which the cement components may be disposed for the mixing process and an agitator further disposed for mixing said cement components

In an embodiment, a mixing apparatus includes a temperature sensing means (also referred to herein as temperature indicator) such as a thermocouple. In an embodiment, a cue (audible, visual, or other such signal) indicates that the mixed cement is no longer pliable enough to be used.

In an embodiment, the mixing apparatus can be operated by a power tool for the mixing of the bone cement. That is, the power tool can be operatively coupled to the agitator and the agitator so attached or coupled to the power tool such that, when the power tool operates, the power tool causes the agitator to rotate or otherwise move within the mixing region.

In an embodiment, the power tool and/or mixing apparatus has a mechanical torque limiter between an input and agitator that may slip, overrun, break or otherwise inhibit the agitator as a result of mixing torque exceeding a threshold value. The mechanical torque limiter can provide a cue (audible, visual, or other) indicating that the mixing phase is complete.

In an embodiment, the apparatus comprises a timer. In another embodiment, the timer of the apparatus may be started by a sensor of the apparatus that determines that the mechanical torque limit has been reached. When the timer is complete, a cue (such as an audible, visual, or other cue) by the apparatus signals to the user that the mixed cement is ready and able to be applied.

In an embodiment, the unmixed bone cement powder and liquid are compartmentalized within the mixing apparatus. In a further embodiment, the inside of the mixing apparatus remains completely sealed and sterile until the bone cement has been mixed and the user is ready to apply said bone cement, at which point an opening in the apparatus is exposed for cement collection.

In another embodiment, a power tool for use with a mixing apparatus is provided. The tool includes a torque limiter that may cease agitation of a mix in the apparatus by the tool once a threshold torque is met. In an embodiment, the torque limiter indicates to the user that a threshold torque has been reached.

### DESCRIPTION OF THE DRAWINGS

The advantages and features of the present disclosure will become better understood with reference to the following detailed description and claims taken in conjunction with the accompanying drawings, wherein like elements are identified with like symbols.

FIG. 1 shows a cross sectional view of a mixing apparatus in accordance with an exemplary embodiment of the present disclosure;

FIG. 2 shows a mixing apparatus wherein cement components are contained within the apparatus prior to mixing in accordance with an exemplary embodiment of the present disclosure.

FIG. 3 shows a mixing apparatus and power tool in accordance with an exemplary embodiment of the present disclosure.

FIG. 4 shows the shearing of a mechanical torque limiting element between an input and the agitator of a mixing apparatus, in accordance with an exemplary embodiment of the present disclosure.

### DETAILED DESCRIPTION OF THE DISCLOSURE

The exemplary embodiments described herein detail for illustrative purposes are subject to many variations in structure and design. That is, it is understood that various omissions and substitutions of equivalents are contemplated as circumstances may suggest or render expedient, but these are intended to cover the application or implementation without departing from the spirit or scope of the claims of the present disclosure. The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

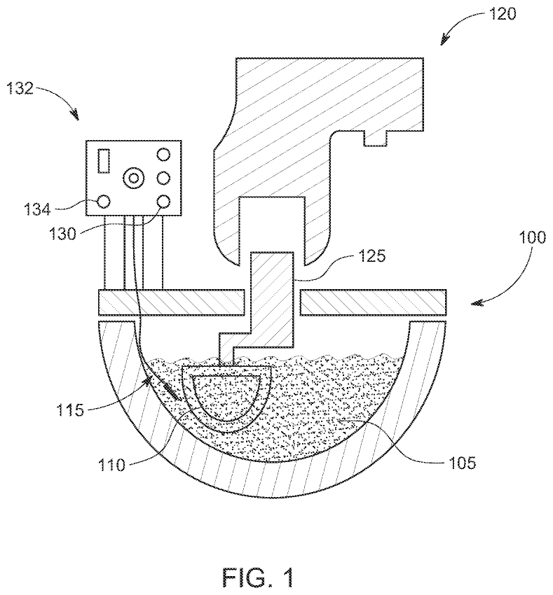

The present disclosure provides for an apparatus 100 for mixing at least two-component cement as shown in FIG. 1, for example, which cement is for use in orthopedic surgeries. In an embodiment the apparatus 100 comprises a mixing region 105 (such as a reservoir, container or bowl, for example) in which the cement components may be disposed for the mixing process. The apparatus 100 comprises an agitator 110 which agitator actuates to mix the cement ingredients (such as, for example, a powdered cement polymer and a liquid monomer) together for forming the cement. In an embodiment, the agitator 110 comprises an impeller blade, however, it will be apparent that the agitator 110 may comprise a whisk, screw, paddle or any other configuration that facilitates mixing of the elements of the cement. In an embodiment, the mixing apparatus 100 uses an asymmetrical mixing pattern to achieve a more uniform mix of a cement product.

Referring again to FIG. 1, in an embodiment, the mixing apparatus 100 includes a temperature sensing means 115 (also referred to herein as temperature indicator or temperature sensor) such as a thermocouple. In an embodiment, a cue (audible, visual, or other such signal) indicates that the cement mix can no longer be used. This cue can come from a circuit board (such as a circuit board of the apparatus 100, for example) that reads the temperature from the temperature sensor 115. In an embodiment the temperature indicator comprises an LED panel that alights when the temperature sensing means 115 reads a temperature at which the cement mix can no longer be used. In an embodiment, the temperature indicator 115 can be a temperature dot or dots disposed on (and therefore easily viewable) the mixing apparatus 100. In a further embodiment, the temperature dot panel may indicate when the mix is approaching being too unpliable to use. In a still further embodiment, the temperature sensing means is integral to the mixing apparatus and further, part of or all of the apparatus can change color with changes in temperature (such as in an embodiment wherein the apparatus comprises a thermochromic material or component.) It should be understood that the behavior of cement is temperature-sensitive such that when the cement's temperature is increased, the working time of the mixture will be comparatively reduced. In an embodiment, the temperature indicator references ambient temperature.

In an embodiment, the temperature indicator 115 is a thermochromic strip. In an embodiment, said thermochromic strip may be predisposed on or in the apparatus 100 to provide a color indication of the temperature of the cement.

In an embodiment, the mixing apparatus 100 has embedded electronics 132 that include buttons, switches, LED(s), a circuit board, and/or microcontroller(s). It will be apparent that such embedded electronics may control and/or be utilized to operate the apparatus 100.

In an embodiment and as shown in FIG. 1, the mixing apparatus 100 can be operated or acted on by a power tool 120 for the mixing of the bone cement. That is, the power tool 120 can be operatively coupled to the agitator 110 and the agitator 110 so attached or coupled to the power tool 120 such that, when the power tool 120 operates (such as in the case of a rotary motion end (e.g. a motorized drill or screwdriver)), the power tool 120 causes the agitator 110 to rotate or otherwise move within the mixing region 105.

In said embodiment, the power tool 120 and/or mixing apparatus 100 has a mechanical torque limiter 125 between an input of the tool 120 and/or apparatus 100 and the agitator 110 that slips, overruns, breaks, indicates or otherwise inhibits the agitator 110 as a result of mixing torque exceeding a threshold value. That is, once the bone cement powder and monomer are in contact with one another, a polymerization reaction occurs and, as a result, the mixture's viscosity starts to increase. This viscosity increase requires an increasing in mixing torque. A threshold torque value is reached at the viscosity that indicates ideal application time of the bone cement. Upon reaching this threshold value, the mechanical torque limiter 125 can provide a cue (audible, visual, or other) indicating that the mixing phase is complete. Such mechanical torque limiter 125 can operate by means of a slip-clutch, for example.

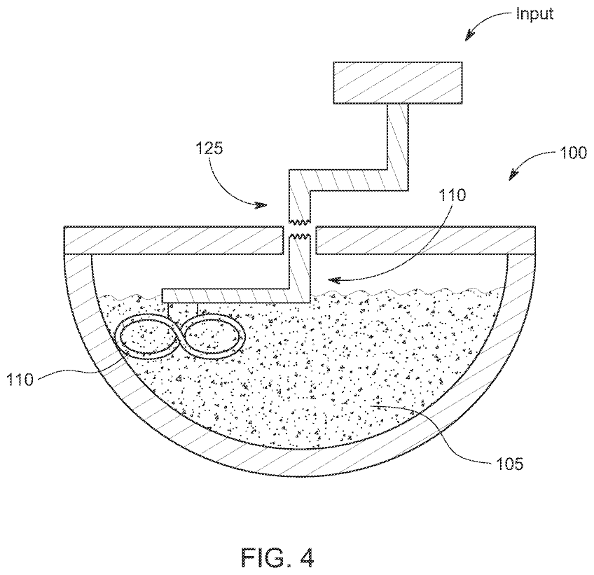

In an embodiment, the apparatus 100 comprises a timer 130, which timer may be incorporated as part of a microcontroller, for example. In an embodiment, the timer 130 of the apparatus 100 may be started by a sensor 134 of the apparatus 100 or by the user in response to a mechanical torque value, for example. When the timer 130 is complete, a cue (such as an audible, visual, or other cue) by the apparatus 100 signals to the user that the mixed cement is ready and able to be applied. In an embodiment the initial countdown setting is controlled by the temperature of the mixed cement. The timer 130 improves outcomes by informing the surgeon and any assistants as to exactly how much time they have until the bone cement is no longer in a state where it is able to be applied. This allows for greater predictability and efficiency in the operating room. In an embodiment, the timer countdown is displayed digitally on the mixing apparatus 100. In a still further embodiment, the mixing apparatus' 100 embedded electronics recognize when the mechanical torque limit has been reached and starts the timer 130. In an embodiment, the mixing apparatus 100 with mechanical torque limiter 125 is manually operated by the user. In a further embodiment, the mechanical torque limiter 125 prevents overmixing by not allowing further mixing after a threshold torque has been reached. This may occur, for example, in the event of an intentional shearing of the connection between the input and the impeller/agitator 110, as shown in FIG. 4 for example.

In an embodiment, the bone cement is ready for use immediately or nearly immediately after mixing is completed. This can be accomplished by configuring the mixing apparatus 100 to only stop mixing when the cement is ready to be applied (i.e., such as when a viscosity of the mixture is reached that indicates ideal application time of the bone cement, such time sometimes commonly referred to as working time).

In an embodiment, the mixing apparatus comprises a means for testing the tackiness of the mix. This could, for example, be a mechanical “finger” that constantly dips into the cement mix measuring the tack of the mixture. Said tackiness measuring means can indicate to the surgeon's assistant/surgeon that the mix is in its dough point and is ready to be applied.

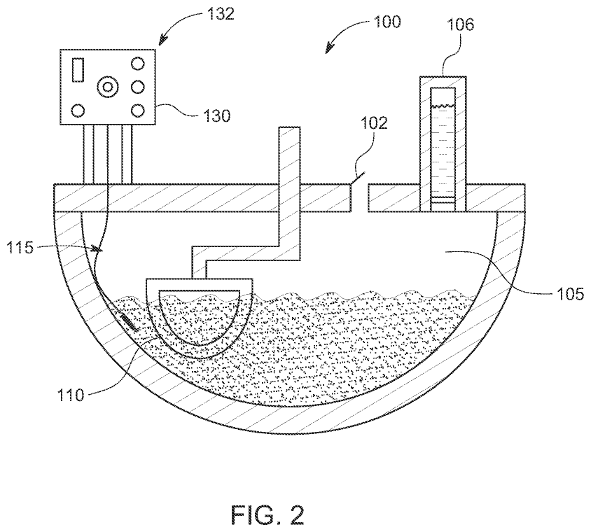

In another embodiment, the unmixed bone cement powder and liquid are contained within the mixing apparatus 100. Prior to mixing, the powder and liquid are preferably contained apart from one another. For example, as shown in FIG. 2, the powder can be sealed within the mixing region 105 (such as the reservoir, for example) of the apparatus 100 while the liquid is contained in a separate compartment 106 of the mixing apparatus 100, which separate compartment 106 is in fluidic communication with the mixing region 105. When the user is ready, he can dispense the liquid into the mixing region 105 (through a selectively openable valve or port that is in communication with the mixing region 105, the opening/closing controls for which may be contained in the apparatus' 100 embedded electronics, for example) in physical proximity to the powder and begin mixing. This allows for clean, sterile, and streamlined mixing.

In a further embodiment, the inside of the mixing apparatus 100 remains completely sealed and sterile until the bone cement has been mixed, with, for example, the liquid being maintained in a sterile condition prior to the dispensing of the liquid into the mixing region 105. This configuration provides the same benefits as mixing under vacuum but eliminates the need for a hose and vacuum pump. In an embodiment, the mixing apparatus 100 contains a one-way valve 102 that prevents pressure buildup when mixing in a sealed environment mixing region 105.

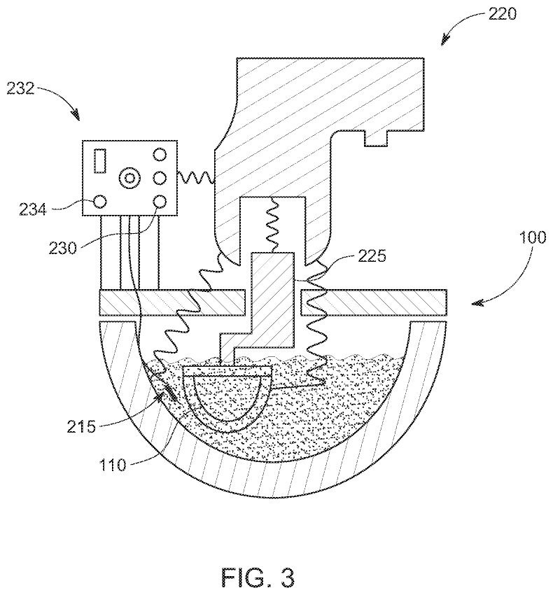

In another embodiment, and as shown in FIG. 3 a power tool 220 for use with a mixing apparatus is provided. The tool is capable of operatively coupling with a mixing apparatus to agitate the contents of the mixing apparatus, such as by having the tool attach to an impeller or other agitating means 110 of the mixing apparatus. The power tool 220 comprise a torque limiter 225 as elsewhere described herein, which torque limiter slips, overruns, breaks or otherwise inhibits the power tool 220 as a result of mixing torque exceeding a threshold value.

In an embodiment, the tool 220 comprises a timer 230, which timer may be incorporated as part of a microcontroller of the tool. In an embodiment, the user presses a button on the power tool 220 after the mechanical torque limit has been reached, which button press may start a timer 230 on the microcontroller. In another embodiment, the timer 230 of the tool 220 may be started by a sensor 234 of the tool 220 that determines that the mechanical torque limit has been reached. When the timer 230 is complete, a cue (such as an audible, visual, or other cue) by the tool 220 signals to the user that the mixing process has completed or reached a particular stage.

In an embodiment, the tool 220 has embedded electronics 232 that include buttons, switches, LED(s), a circuit board, and/or microcontroller(s). It will be apparent that such embedded electronics may control and/or be utilized to operate the tool 220.

In an embodiment, the tool 220 includes a temperature sensing means 215 (also referred to herein as temperature indicator) such as a thermocouple, which temperature sensing means may operatively couple to a mixing apparatus to sense temperature of or within the mixing apparatus. In an embodiment, a cue (audible, visual, or other such signal) indicates that a certain temperature has been reached in the mixing apparatus. This cue can come from a circuit board (such as a circuit board of the tool 220, for example) that reads the temperature from the temperature indicator 215. In an embodiment, the temperature indicator 215 can be a temperature dot or dots disposed on (and therefore easily viewable) on the tool 220. In an embodiment the temperature indicator comprises an LED panel that alights when the temperature sensing means 215 reads a temperature at which the cement mix cannot be used any longer. In a further embodiment, the temperature dot panel may indicate when a mix in the apparatus is approaching being too unpliable to use.

In an embodiment, the mechanical torque limiter may cause a decoupling of the agitator when a particular torque limit is met. In an exemplary embodiment, and as shown in FIG. 4, the coupling between an input (such as a power tool or a handle otherwise coupled to the agitator) and the agitator shears when a torque limit is reached. This embodiment has an advantage of providing an easily discernable visual, audible and/or physical cue that the cement has reached the condition in which it is in its working state.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the disclosure and various embodiments with various modifications as are suited to the particular use contemplated.

What is claimed is:

1. An apparatus for mixing at least a two-component bone cement, the apparatus comprising
a mixing region,
an agitator,
a means for actuating the agitator,
a mechanical torque limiter,
wherein the mixing region receives at least one cement ingredient and said agitator mixes said at least one cement ingredient to form a cement and wherein said torque limiter is disposed between said agitator and said means for actuating the actuator.

2. The apparatus of claim 1, wherein said agitator is no longer actuated when the mechanical torque limiting device reaches a predetermined value.

3. The apparatus of claim 2, wherein the predetermined value is at least two times the initial mixing torque.

4. The apparatus of claim 2, wherein the agitator and means for actuating the agitator operatively decouple when the mechanical torque limiting device reaches the predetermined value.

5. The apparatus of claim 2, wherein the agitator and means for actuating the agitator physically decouple when the mechanical torque limiting device reaches the predetermined value.

6. The apparatus of claim 1, the apparatus further comprising a temperature sensing means, said temperature sensing means capable of sensing and signaling at least one of a cement mix in the mixing apparatus can no longer be used and the temperature of a mixture in the mixing region of the apparatus.

7. The apparatus of claim 1, wherein the means for actuating the agitator further comprises a power tool.

8. The apparatus of claim 1, wherein said torque limiter is capable of sensing and signaling that a threshold torque of the apparatus has been reached.

9. The apparatus of claim 8, said apparatus further comprising a timer, and wherein said timer is initiated after said torque limiter has sensed that a threshold torque of the apparatus has been reached.

10. The apparatus of claim 9, wherein the mixing region remains completely sealed and sterile until the cement has been mixed.

11. The apparatus of claim 10, said mixing region comprising a one-way valve that prevents pressure buildup when mixing in a sealed environment.

12. A power tool fora bone cement mixing apparatus, the mixing apparatus comprising an agitator, the power tool operatively coupled to and of selectively actuating the agitator of the mixing apparatus, and the power tool comprising a mechanical torque limiter, which mechanical torque limiter may cause a selective and temporary disengagement of the power tool from the agitator.

13. The power tool of claim 12, wherein said power tool ceases actuation when the mechanical torque limiting device reaches a predetermined value.

14. The power tool of claim 13, wherein the predetermined value is at least two times the initial mixing torque.

15. The power tool of claim 12, wherein said mechanical torque limiter is capable of sensing and signaling at least one of that a mixing phase of the apparatus is complete, that a threshold torque of the tool has been reached, and that no further mixing occurs after a certain torque has been reached.

16. The power tool of claim 12, said tool further comprising a timer, and wherein said timer is initiated after said torque limiter has sensed that a threshold torque of the tool has been reached.

17. The power tool of claim 13, wherein the power tool and agitator operatively decouple when the mechanical torque limiting device reaches the predetermined value.

18. The power tool of claim 13, wherein the power tool and agitator physically decouple when the mechanical torque limiting device reaches the predetermined value.

19. An apparatus for mixing at least a two-component bone cement, the apparatus comprising
a mixing region, said mixing region comprising a temperature sensing means
an agitator,
a means for actuating the agitator,
wherein the mixing region receives at least one cement ingredient and said agitator mixes said at least one cement ingredient to form a cement and wherein said temperature sensing means provides indication of a condition of the cement.

20. The apparatus of claim 19 further comprising a mechanical torque limiter disposed between said agitator and said means for actuating the actuator.
